(19) United States
(12) Patent Application Publication
(10) Pub. No.: US 2026/0295007 A1
(43) Pub. Date: Oct. 1, 2026

(54) BETA-CASEINS IN INFANT MILK FORMULA AND USE FOR REDUCING REGURGITATION EPISODES IN INFANTS

(71) Applicant: THE A2 MILK COMPANY LIMITED, Auckland (NZ)

(72) Inventors: Andrew John Clarke, Auckland (NZ); Julia Zhu, Auckland (NZ)

(21) Appl. No.: 19/478,677

(22) PCT Filed: Apr. 24, 2024
(86) PCT No.: PCT/NZ2024/050043

§ 371 Date: Oct. 24, 2025

Related U.S. Application Data
(60) Provisional application No. 63/461,721, filed on Apr. 25, 2023.

(51) Int. Cl. A61K 38/17; A23L 33/00; A61K 35/20; A61P 1/08

(52) U.S. Cl. CPC A61K 38/1709; A23L 33/40; A61K 35/20; A61P 1/08

(57) ABSTRACT

Reducing the frequency of regurgitation episodes in an infant by providing infant milk formula manufactured from milk which comprises beta-casein, wherein the beta-casein comprises less than 30% by weight of beta-caseins having histidine at position 67 of the beta-casein amino acid sequence.

### TECHNICAL FIELD

The invention relates to the milk protein beta-casein and reducing the frequency or duration of regurgitation episodes in infants. In particular, the invention relates to infant milk formula made with milk having a beta-casein profile that comprises little or no beta-casein A1 or related beta-casein variants. The applicant has found that the consumption of infant milk formula made with milk containing beta-casein variants that readily produce BCM-7 on digestion is associated with increased regurgitation episodes in infants relative to infant milk formula made with milk containing beta-casein variants that do not readily produce BCM-7 on digestion.

### BACKGROUND OF THE INVENTION

The benefits to infants of breast feeding for at least the first six months of life, and preferably for another 6 to 12 months, are well-established. Human breast milk is known to protect infants from infections and to reduce the rates of health problems occurring including diabetes, obesity and asthma. It is widely accepted that the entire intestinal flora of breast-fed infants provides anti-infective properties and is an important stimulating factor for the postnatal development of the immune system. Breast milk is universally regarded as the best source of nutrition for a new baby. However, it is also well-known that many mothers are not able to breast feed their babies and therefore the use of infant milk formula (also known as infant formula or milk formula) to feed babies is the preferred or, in some cases, the only option.

Mature human milk contains 3-5% fat, 0.8-0.9% protein, 6.9-7.2% carbohydrate (calculated as lactose), and 0.2% mineral constituents. The principal human milk proteins are whey and casein. The concentration of whey proteins decreases from early lactation and continues to fall. These changes result in a whey/casein ratio of about 90:10 in early lactation, 60:40 in mature milk and 50:50 in late lactation. The principal proteins of human milk are beta-casein, alpha-lactalbumin, lactoferrin, immunoglobulin IgA, lysozyme, and serum albumin. The essential amino acid pattern of human milk closely resembles that found to be optimal for human infants.

The composition of infant milk formula is designed to be based on human mother's milk at approximately one to three months postpartum. The protein component of the most commonly used infant milk formulae comprises casein and whey from bovine milk plus added purified whey. They also contain a blend of vegetable oils as a fat source, lactose as a carbohydrate source, a vitamin-mineral mix, and other ingredients depending on the manufacturer. In addition, some infant milk formulae use soybean as a protein source instead of bovine milk and some infant milk formulae use protein hydrolysed into its component amino acids for infants who are allergic to other proteins. Besides human breast milk, infant milk formula is the only other milk product that the medical community considers to be nutritionally acceptable for infants under the age of one year (in contrast to cows' milk, goat milk, or follow-on formulae of varied compositions).

Bovine milk typically comprises around 30 grams per litre of protein. Caseins make up the largest component (80%) of that protein, and beta-caseins make up about 37% of the caseins. In the past three decades, the body of evidence implicating casein proteins, especially beta-caseins, in a number of health disorders has been growing.

The beta-casein family comprises several variants, which are routinely known as A1, A2, A3, B, C, D, E, F, G, H1, H2 and I. There may also be other variants not yet identified. Beta-casein A1 and beta-casein A2 are the predominant beta-caseins in milk consumed in most human populations. The applicant and others have previously determined a link between the consumption of beta-casein A1 in milk and milk products and the incidence of certain health conditions including type I diabetes (WO 1996/014577), coronary heart disease (WO 1996/036239) and neurological disorders (WO 2002/019832). Further, the applicant has shown a link between beta-casein A1 and bowel inflammation (WO 2014/193248), lactose intolerance (WO 2015/005804), high blood glucose levels (WO 2015/026245), antioxidant capacity (WO 2016/190750), cognitive function (WO 2017/171563) and gut microbiota (WO 2018/063008).

Beta-casein A1 differs from beta-casein A2 by a single amino acid. A histidine amino acid is located at position 67 of the 209 amino acid sequence of beta-casein A1, whereas a proline is located at the same position of beta-casein A2. This single amino acid difference is, however, critically important to the enzymatic digestion of beta-caseins in the gut. The presence of histidine at position 67 allows a protein fragment comprising seven amino acids, known as beta-casomorphin-7 (BCM-7), to be produced readily on enzymatic digestion. Thus, BCM-7 is a digestion product of beta-casein A1. In the case of beta-casein A2, position 67 is occupied by a proline which hinders cleavage of the protein at that location. Thus, BCM-7 is not produced readily on digestion of beta-casein A2.

Other beta-casein variants, such as the B, C, F, G, H1 and H2 variants, also have histidine at position 67, whereas variants in addition to the A2 variant, such as the A3, D, E and I variants, have proline at position 67.

BCM-7 is an opioid peptide and can bind to and activate opioid receptors throughout the body. BCM-7 is able to cross the gastrointestinal wall and enter circulation enabling it to influence systemic and cellular activities via opioid receptors.

The applicant has now found an association between infant milk formula containing beta-casein variants having histidine at position 67 of the beta-casein amino acid sequence and a higher frequency of regurgitation episodes in infants relative to infant milk formula containing variants having proline at position 67 of the beta-casein amino acid sequence. This effect is considered to be caused by the BCM-7 produced on digestion of beta-casein A1, and related variants, in the gut. Therefore, any beta-casein variant having histidine at position 67, and capable of readily producing BCM-7 on digestion, in the diet of infants is undesirable.

It is an object of the invention to provide a method for reducing the frequency of regurgitation episodes in an infant, or to at least provide a useful alternative to existing methods.

### SUMMARY OF THE INVENTION

In a first aspect of the invention there is provided a method for reducing the frequency of regurgitation episodes in an infant by providing infant milk formula to the infant for ingestion, wherein the milk used in the manufacture of the infant milk formula contains beta-casein comprising less than 30% by weight of beta-caseins having histidine at position 67 of the beta-casein amino acid sequence.

In certain embodiments of this aspect of the invention, the beta-casein comprises less than 30% by weight of any one or more of beta-casein variants A1, B, C, F, G, H1 and H2.

In certain embodiments of this aspect of the invention, the beta-casein comprises less than 25% by weight of any one or more of beta-casein variants A1, B, C, F, G, H1 and H2, less than 10% by weight of any one or more of beta-casein variants A1, B, C, F, G, H1 and H2, less than 5% by weight of any one or more of beta-casein variants A1, B, C, F, G, H1 and H2, less than 1% by weight of any one or more of beta-casein variants A1, B, C, F, G, H1 and H2, or none of beta-casein variants A1, B, C, F, G, H1 and H2.

In certain embodiments of this aspect of invention, the beta-casein comprises less than 30% by weight of beta-casein A1.

In certain embodiments of this aspect of the invention, the beta-casein comprises less than 25% by weight beta-casein A1, less than 10% by weight beta-casein A1, less than 5% by weight beta-casein A1, less than 1% by weight beta-casein A1, or no beta-casein A1.

In certain embodiments of this aspect of the invention, the beta-casein comprises at least 70% by weight of beta-caseins having proline at position 67 of the beta-casein amino acid sequence.

In certain embodiments of this aspect of the invention, the beta-casein comprises at least 70% by weight of any one or more of beta-casein variants A2, A3, D, E and I, at least 75% by weight of any one or more of beta-casein variants A2, A3, D, E and I, at least 90% by weight of any one or more of beta-casein variants A2, A3, D, E and I, at least 95% by weight of any one or more of beta-casein variants A2, A3, D, E and I, at least 99% by weight of any one or more of beta-casein variants A2, A3, D, E and I, or 100% by weight of any one or more of beta-casein variants A2, A3, D, E and I.

In certain embodiments of this aspect of the invention, the beta-casein comprises at least 70% by weight beta-casein A2, at least 75% by weight beta-casein A2, at least 90% by weight beta-casein A2, at least 95% by weight beta-casein A2, at least 99% by weight beta-casein A2, or 100% beta-casein A2.

In preferred embodiments of this aspect of the invention, the infant milk formula additionally comprises fat, lactose, minerals and vitamins.

In certain embodiments of this aspect of the invention, the infant has an age less than six months, or less than three months.

In a second aspect of the invention there is provided a method for reducing the frequency of regurgitation episodes in an infant by feeding infant milk formula to the infant, wherein the milk used in the manufacture of the infant milk formula contains beta-casein comprising less than 30% by weight of beta-caseins having histidine at position 67 of the beta-casein amino acid sequence.

In certain embodiments of this aspect of the invention, the beta-casein comprises less than 30% by weight of any one or more of beta-casein variants A1, B, C, F, G, H1 and H2.

In certain embodiments of this aspect of the invention, the beta-casein comprises less than 25% by weight of any one or more of beta-casein variants A1, B, C, F, G, H1 and H2, less than 10% by weight of any one or more of beta-casein variants A1, B, C, F, G, H1 and H2, less than 5% by weight of any one or more of beta-casein variants A1, B, C, F, G, H1 and H2, less than 1% by weight of any one or more of beta-casein variants A1, B, C, F, G, H1 and H2, or none of beta-casein variants A1, B, C, F, G, H1 and H2.

In certain embodiments of this aspect of invention, the beta-casein comprises less than 30% by weight of beta-casein A1.

In certain embodiments of this aspect of the invention, the beta-casein comprises less than 25% by weight beta-casein A1, less than 10% by weight beta-casein A1, less than 5% by weight beta-casein A1, less than 1% by weight beta-casein A1, or no beta-casein A1.

In certain embodiments of this aspect of the invention, the beta-casein comprises at least 70% by weight of beta-caseins having proline at position 67 of the beta-casein amino acid sequence.

In certain embodiments of this aspect of the invention, the beta-casein comprises at least 70% by weight of any one or more of beta-casein variants A2, A3, D, E and I, at least 75% by weight of any one or more of beta-casein variants A2, A3, D, E and I, at least 90% by weight of any one or more of beta-casein variants A2, A3, D, E and I, at least 95% by weight of any one or more of beta-casein variants A2, A3, D, E and I, at least 99% by weight of any one or more of beta-casein variants A2, A3, D, E and I, or 100% by weight of any one or more of beta-casein variants A2, A3, D, E and I.

In certain embodiments of this aspect of the invention, the beta-casein comprises at least 70% by weight beta-casein A2, at least 75% by weight beta-casein A2, at least 90% by weight beta-casein A2, at least 95% by weight beta-casein A2, at least 99% by weight beta-casein A2, or 100% beta-casein A2.

In preferred embodiments of this aspect of the invention, the infant milk formula additionally comprises fat, lactose, minerals and vitamins.

In certain embodiments of this aspect of the invention, the infant has an age less than six months, or less than three months.

In another aspect of the invention there is provided a method for reducing the frequency of regurgitation episodes in an infant having a diet comprising infant milk formula where the infant milk formula contains beta-casein comprising at least 30% by weight of beta-caseins having histidine at position 67 of the beta-casein amino acid sequence, by replacing the infant milk formula with a replacement infant milk formula manufactured from milk containing beta-casein comprising less than 30% by weight of beta-caseins having histidine at position 67 of the beta-casein amino acid sequence.

In certain embodiments of this aspect of the invention, the beta-casein of the replacement infant milk formula comprises less than 30% by weight of any one or more of beta-casein variants A1, B, C, F, G, H1 and H2.

In certain embodiments of this aspect of the invention, the beta-casein of the replacement infant milk formula comprises less than 25% by weight of any one or more of beta-casein variants A1, B, C, F, G, H1 and H2, less than 10% by weight of any one or more of beta-casein variants A1, B, C, F, G, H1 and H2, less than 5% by weight of any one or more of beta-casein variants A1, B, C, F, G, H1 and H2, less than 1% by weight of any one or more of beta-casein variants A1, B, C, F, G, H1 and H2, or none of beta-casein variants A1, B, C, F, G, H1 and H2.

In certain embodiments of this aspect of invention, the beta-casein of the replacement infant milk formula comprises less than 30% by weight of beta-casein A1.

In certain embodiments of this aspect of the invention, the beta-casein of the replacement infant milk formula comprises less than 25% by weight beta-casein A1, less than 10% by weight beta-casein A1, less than 5% by weight beta-casein A1, less than 1% by weight beta-casein A1, or no beta-casein A1.

In certain embodiments of this aspect of the invention, the beta-casein of the replacement infant milk formula comprises at least 70% by weight of beta-caseins having proline at position 67 of the beta-casein amino acid sequence.

In certain embodiments of this aspect of the invention, the beta-casein comprises at least 70% by weight of any one or more of beta-casein variants A2, A3, D, E and I, at least 75% by weight of any one or more of beta-casein variants A2, A3, D, E and I, at least 90% by weight of any one or more of beta-casein variants A2, A3, D, E and I, at least 95% by weight of any one or more of beta-casein variants A2, A3, D, E and I, at least 99% by weight of any one or more of beta-casein variants A2, A3, D, E and I, or 100% by weight of any one or more of beta-casein variants A2, A3, D, E and I.

In certain embodiments of this aspect of the invention, the beta-casein of the replacement infant milk formula comprises at least 70% by weight beta-casein A2, at least 75% by weight beta-casein A2, at least 90% by weight beta-casein A2, at least 95% by weight beta-casein A2, at least 99% by weight beta-casein A2, or 100% beta-casein A2.

In preferred embodiments of this aspect of the invention, the replacement infant milk formula additionally comprises fat, lactose, minerals and vitamins.

In certain embodiments of this aspect of the invention, the infant has an age less than six months, or less than three months.

In another aspect of the invention there is provided a method for reducing the frequency of regurgitation episodes in an infant having a diet comprising a first infant milk formula, wherein the first infant milk formula is replaced with a second infant milk formula in the diet of the infant;
wherein the first infant milk formula contains beta-casein comprising at least 30% by weight of beta-caseins having histidine at position 67 of the beta-casein amino acid sequence; and
wherein the second infant milk formula is manufactured from milk containing beta-casein comprising less than 30% by weight of beta-caseins having histidine at position 67 of the beta-casein amino acid sequence.

In certain embodiments of this aspect of the invention, the beta-casein of the second infant milk formula is manufactured from milk containing beta-casein comprising less than 30% by weight of any one or more of beta-casein variants A1, B, C, F, G, H1 and H2.

In certain embodiments of this aspect of the invention, the second infant milk formula is manufactured from milk containing beta-casein comprising less than 25% by weight of any one or more of beta-casein variants A1, B, C, F, G, H1 and H2, less than 10% by weight of any one or more of beta-casein variants A1, B, C, F, G, H1 and H2, less than 5% by weight of any one or more of beta-casein variants A1, B, C, F, G, H1 and H2, less than 1% by weight of any one or more of beta-casein variants A1, B, C, F, G, H1 and H2, or none of beta-casein variants A1, B, C, F, G, H1 and H2.

In certain embodiments of this aspect of invention, the second infant milk formula is manufactured from milk containing beta-casein comprising less than 30% by weight of beta-casein A1.

In certain embodiments of this aspect of the invention, the second infant milk formula is manufactured from milk containing beta-casein comprising less than 25% by weight beta-casein A1, less than 10% by weight beta-casein A1, less than 5% by weight beta-casein A1, less than 1% by weight beta-casein A1, or no beta-casein A1.

In certain embodiments of this aspect of the invention, the second infant milk formula is manufactured from milk containing beta-casein comprising at least 70% by weight of beta-caseins having proline at position 67 of the beta-casein amino acid sequence.

In certain embodiments of this aspect of the invention, the beta-casein comprises at least 70% by weight of any one or more of beta-casein variants A2, A3, D, E and I, at least 75% by weight of any one or more of beta-casein variants A2, A3, D, E and I, at least 90% by weight of any one or more of beta-casein variants A2, A3, D, E and I, at least 95% by weight of any one or more of beta-casein variants A2, A3, D, E and I, at least 99% by weight of any one or more of beta-casein variants A2, A3, D, E and I, or 100% by weight of any one or more of beta-casein variants A2, A3, D, E and I.

In certain embodiments of this aspect of the invention, the second infant milk formula is manufactured from milk containing beta-casein comprising at least 70% by weight beta-casein A2, at least 75% by weight beta-casein A2, at least 90% by weight beta-casein A2, at least 95% by weight beta-casein A2, at least 99% by weight beta-casein A2, or 100% beta-casein A2.

In preferred embodiments of this aspect of the invention, the infant milk formula additionally comprises fat, lactose, minerals and vitamins.

In certain embodiments of this aspect of the invention, the infant has an age less than six months, or less than three months.

In another aspect of the invention there is provided infant milk formula for reducing the frequency of regurgitation episodes in an infant which infant milk formula is manufactured from milk containing beta-casein comprising less than 30% by weight of beta-caseins having histidine at position 67 of the beta-casein amino acid sequence.

In certain embodiments of this aspect of the invention, the beta-casein comprises less than 30% by weight of any one or more of beta-casein variants A1, B, C, F, G, H1 and H2.

In certain embodiments of this aspect of the invention, the beta-casein comprises less than 25% by weight of any one or more of beta-casein variants A1, B, C, F, G, H1 and H2, less than 10% by weight of any one or more of beta-casein variants A1, B, C, F, G, H1 and H2, less than 5% by weight of any one or more of beta-casein variants A1, B, C, F, G, H1 and H2, less than 1% by weight of any one or more of beta-casein variants A1, B, C, F, G, H1 and H2, or none of beta-casein variants A1, B, C, F, G, H1 and H2.

In certain embodiments of this aspect of invention, the beta-casein comprises less than 30% by weight of beta-casein A1.

In certain embodiments of this aspect of the invention, the beta-casein comprises less than 25% by weight beta-casein A1, less than 10% by weight beta-casein A1, less than 5% by weight beta-casein A1, less than 1% by weight beta-casein A1, or no beta-casein A1.

In certain embodiments of this aspect of the invention, the beta-casein comprises at least 70% by weight of beta-caseins having proline at position 67 of the beta-casein amino acid sequence.

In certain embodiments of this aspect of the invention, the beta-casein comprises at least 70% by weight of any one or more of beta-casein variants A2, A3, D, E and I, at least 75% by weight of any one or more of beta-casein variants A2, A3, D, E and I, at least 90% by weight of any one or more of beta-casein variants A2, A3, D, E and I, at least 95% by weight of any one or more of beta-casein variants A2, A3, D, E and I, at least 99% by weight of any one or more of beta-casein variants A2, A3, D, E and I, or 100% by weight of any one or more of beta-casein variants A2, A3, D, E and I.

In certain embodiments of this aspect of the invention, the beta-casein comprises at least 70% by weight beta-casein A2, at least 75% by weight beta-casein A2, at least 90% by weight beta-casein A2, at least 95% by weight beta-casein A2, at least 99% by weight beta-casein A2, or 100% beta-casein A2.

In preferred embodiments of this aspect of the invention, the infant milk formula additionally comprises fat, lactose, minerals and vitamins.

In certain embodiments of this aspect of the invention, the infant has an age less than six months, or less than three months.

In another aspect of the invention there is provided the use of bovine milk in the manufacture of infant milk formula for reducing the frequency of regurgitation episodes in an infant, wherein the milk contains beta-casein comprising less than 30% by weight of beta-caseins having histidine at position 67 of the beta-casein amino acid sequence.

In certain embodiments of this aspect of the invention, the beta-casein comprises less than 30% by weight of any one or more of beta-casein variants A1, B, C, F, G, H1 and H2.

In certain embodiments of this aspect of the invention, the beta-casein comprises less than 25% by weight of any one or more of beta-casein variants A1, B, C, F, G, H1 and H2, less than 10% by weight of any one or more of beta-casein variants A1, B, C, F, G, H1 and H2, less than 5% by weight of any one or more of beta-casein variants A1, B, C, F, G, H1 and H2, less than 1% by weight of any one or more of beta-casein variants A1, B, C, F, G, H1 and H2, or none of beta-casein variants A1, B, C, F, G, H1 and H2.

In certain embodiments of this aspect of invention, the beta-casein comprises less than 30% by weight of beta-casein A1.

In certain embodiments of this aspect of the invention, the beta-casein comprises less than 25% by weight beta-casein A1, less than 10% by weight beta-casein A1, less than 5% by weight beta-casein A1, less than 1% by weight beta-casein A1, or no beta-casein A1.

In certain embodiments of this aspect of the invention, the beta-casein comprises at least 70% by weight of beta-caseins having proline at position 67 of the beta-casein amino acid sequence.

In certain embodiments of this aspect of the invention, the beta-casein comprises at least 70% by weight of any one or more of beta-casein variants A2, A3, D, E and I, at least 75% by weight of any one or more of beta-casein variants A2, A3, D, E and I, at least 90% by weight of any one or more of beta-casein variants A2, A3, D, E and I, at least 95% by weight of any one or more of beta-casein variants A2, A3, D, E and I, at least 99% by weight of any one or more of beta-casein variants A2, A3, D, E and I, or 100% by weight of any one or more of beta-casein variants A2, A3, D, E and I.

In certain embodiments of this aspect of the invention, the beta-casein comprises at least 70% by weight beta-casein A2, at least 75% by weight beta-casein A2, at least 90% by weight beta-casein A2, at least 95% by weight beta-casein A2, at least 99% by weight beta-casein A2, or 100% beta-casein A2.

In preferred embodiments of this aspect of the invention, the infant milk formula additionally comprises fat, lactose, minerals and vitamins.

In certain embodiments of this aspect of the invention, the infant has an age less than six months, or less than three months.

In yet another aspect of the invention there is provided a method for reducing the frequency of regurgitation episodes in an infant by providing infant milk formula to the infant for ingestion, wherein the infant milk formula contains beta-casein comprising less than 30% by weight of beta-caseins having histidine at position 67 of the beta-casein amino acid sequence.

In certain embodiments of this aspect of the invention, the beta-casein comprises less than 30% by weight of any one or more of beta-casein variants A1, B, C, F, G, H1 and H2.

In certain embodiments of this aspect of the invention, the beta-casein comprises less than 25% by weight of any one or more of beta-casein variants A1, B, C, F, G, H1 and H2, less than 10% by weight of any one or more of beta-casein variants A1, B, C, F, G, H1 and H2, less than 5% by weight of any one or more of beta-casein variants A1, B, C, F, G, H1 and H2, less than 1% by weight of any one or more of beta-casein variants A1, B, C, F, G, H1 and H2, or none of beta-casein variants A1, B, C, F, G, H1 and H2.

In certain embodiments of this aspect of invention, the beta-casein comprises less than 30% by weight of beta-casein A1.

In certain embodiments of this aspect of the invention, the beta-casein comprises less than 25% by weight beta-casein A1, less than 10% by weight beta-casein A1, less than 5% by weight beta-casein A1, less than 1% by weight beta-casein A1, or no beta-casein A1.

In certain embodiments of this aspect of the invention, the beta-casein comprises at least 70% by weight of beta-caseins having proline at position 67 of the beta-casein amino acid sequence.

In certain embodiments of this aspect of the invention, the beta-casein comprises at least 70% by weight of any one or more of beta-casein variants A2, A3, D, E and I, at least 75% by weight of any one or more of beta-casein variants A2, A3, D, E and I, at least 90% by weight of any one or more of beta-casein variants A2, A3, D, E and I, at least 95% by weight of any one or more of beta-casein variants A2, A3, D, E and I, at least 99% by weight of any one or more of beta-casein variants A2, A3, D, E and I, or 100% by weight of any one or more of beta-casein variants A2, A3, D, E and I.

In certain embodiments of this aspect of the invention, the beta-casein comprises at least 70% by weight beta-casein A2, at least 75% by weight beta-casein A2, at least 90% by weight beta-casein A2, at least 95% by weight beta-casein A2, at least 99% by weight beta-casein A2, or 100% beta-casein A2.

In preferred embodiments of this aspect of the invention, the infant milk formula additionally comprises fat, lactose, minerals and vitamins.

In certain embodiments of this aspect of the invention, the infant has an age less than six months, or less than three months.

In another aspect of the invention there is provided a method for reducing the frequency of regurgitation episodes in an infant having a diet comprising infant milk formula where the infant milk formula contains beta-casein comprising at least 30% by weight of beta-caseins having histidine at position 67 of the beta-casein amino acid sequence, by replacing the infant milk formula with a replacement infant milk formula containing beta-casein comprising less than 30% by weight of beta-caseins having histidine at position 67 of the beta-casein amino acid sequence.

In certain embodiments of this aspect of the invention, the beta-casein of the replacement infant milk formula comprises less than 30% by weight of any one or more of beta-casein variants A1, B, C, F, G, H1 and H2.

In certain embodiments of this aspect of the invention, the beta-casein of the replacement infant milk formula comprises less than 25% by weight of any one or more of beta-casein variants A1, B, C, F, G, H1 and H2, less than 10% by weight of any one or more of beta-casein variants A1, B, C, F, G, H1 and H2, less than 5% by weight of any one or more of beta-casein variants A1, B, C, F, G, H1 and H2, less than 1% by weight of any one or more of beta-casein variants A1, B, C, F, G, H1 and H2, or none of beta-casein variants A1, B, C, F, G, H1 and H2.

In certain embodiments of this aspect of invention, the beta-casein of the replacement infant milk formula comprises less than 30% by weight of beta-casein A1.

In certain embodiments of this aspect of the invention, the beta-casein of the replacement infant milk formula comprises less than 25% by weight beta-casein A1, less than 10% by weight beta-casein A1, less than 5% by weight beta-casein A1, less than 1% by weight beta-casein A1, or no beta-casein A1.

In certain embodiments of this aspect of the invention, the beta-casein of the replacement infant milk formula comprises at least 70% by weight of beta-caseins having proline at position 67 of the beta-casein amino acid sequence.

In certain embodiments of this aspect of the invention, the beta-casein comprises at least 70% by weight of any one or more of beta-casein variants A2, A3, D, E and I, at least 75% by weight of any one or more of beta-casein variants A2, A3, D, E and I, at least 90% by weight of any one or more of beta-casein variants A2, A3, D, E and I, at least 95% by weight of any one or more of beta-casein variants A2, A3, D, E and I, at least 99% by weight of any one or more of beta-casein variants A2, A3, D, E and I, or 100% by weight of any one or more of beta-casein variants A2, A3, D, E and I.

In certain embodiments of this aspect of the invention, the beta-casein of the replacement infant milk formula comprises at least 70% by weight beta-casein A2, at least 75% by weight beta-casein A2, at least 90% by weight beta-casein A2, at least 95% by weight beta-casein A2, at least 99% by weight beta-casein A2, or 100% beta-casein A2.

In preferred embodiments of this aspect of the invention, the replacement infant milk formula additionally comprises fat, lactose, minerals and vitamins.

In certain embodiments of this aspect of the invention, the infant has an age less than six months, or less than three months.

In another aspect of the invention there is provided a method for reducing the frequency of regurgitation episodes in an infant having a diet comprising a first infant milk formula, wherein the first infant milk formula is replaced with a second infant milk formula in the diet of the infant;
wherein the first infant milk formula contains beta-casein comprising at least 30% by weight of beta-caseins having histidine at position 67 of the beta-casein amino acid sequence; and
wherein the second infant milk formula contains beta-casein comprising less than 30% by weight of beta-caseins having histidine at position 67 of the beta-casein amino acid sequence.

In certain embodiments of this aspect of the invention, the beta-casein of the second infant milk formula comprises less than 30% by weight of any one or more of beta-casein variants A1, B, C, F, G, H1 and H2.

In certain embodiments of this aspect of the invention, the second infant milk formula contains beta-casein comprising less than 25% by weight of any one or more of beta-casein variants A1, B, C, F, G, H1 and H2, less than 10% by weight of any one or more of beta-casein variants A1, B, C, F, G, H1 and H2, less than 5% by weight of any one or more of beta-casein variants A1, B, C, F, G, H1 and H2, less than 1% by weight of any one or more of beta-casein variants A1, B, C, F, G, H1 and H2, or none of beta-casein variants A1, B, C, F, G, H1 and H2.

In certain embodiments of this aspect of invention, the second infant milk formula contains beta-casein comprising less than 30% by weight of beta-casein A1.

In certain embodiments of this aspect of the invention, the second infant milk formula contains beta-casein comprising less than 25% by weight beta-casein A1, less than 10% by weight beta-casein A1, less than 5% by weight beta-casein A1, less than 1% by weight beta-casein A1, or no beta-casein A1.

In certain embodiments of this aspect of the invention, the second infant milk formula contains beta-casein comprising at least 70% by weight of beta-caseins having proline at position 67 of the beta-casein amino acid sequence.

In certain embodiments of this aspect of the invention, the beta-casein comprises at least 70% by weight of any one or more of beta-casein variants A2, A3, D, E and I, at least 75% by weight of any one or more of beta-casein variants A2, A3, D, E and I, at least 90% by weight of any one or more of beta-casein variants A2, A3, D, E and I, at least 95% by weight of any one or more of beta-casein variants A2, A3, D, E and I, at least 99% by weight of any one or more of beta-casein variants A2, A3, D, E and I, or 100% by weight of any one or more of beta-casein variants A2, A3, D, E and I.

In certain embodiments of this aspect of the invention, the second infant milk formula contains beta-casein comprising at least 70% by weight beta-casein A2, at least 75% by weight beta-casein A2, at least 90% by weight beta-casein A2, at least 95% by weight beta-casein A2, at least 99% by weight beta-casein A2, or 100% beta-casein A2.

In preferred embodiments of this aspect of the invention, the infant milk formula additionally comprises fat, lactose, minerals and vitamins.

In certain embodiments of this aspect of the invention, the infant has an age less than six months, or less than three months.

### BRIEF DESCRIPTION OF THE FIGURES

FIG. 1 is a graph showing average daily spitting up/vomiting (regurgitation) episodes.

FIG. 2 is a graph showing Infant Gastrointestinal Symptom Questionnaire (IGSQ) composite scores.

### DETAILED DESCRIPTION

The term “infant” means a child under the age of 12 months.

The term “infant milk formula” means a composition for ingestion by an infant in place of or in addition to human milk, and is as defined by the United States Federal Food, Drug, and Cosmetic Act as a “food which purports to be or is represented for special dietary use solely as a food for infants by reason of its simulation of human milk or its suitability as a complete or partial substitute for human milk”, Infant milk formula will typically be in powdered form which is reconstituted in water before feeding to an infant.

The term “beta-casomorphin-7” or “BCM-7” refers to the protein fragment Tyr-Pro-Phe-Pro-Gly-Pro-Ile, a heptapeptide produced readily on enzymatic digestion of bovine beta-casein variants that have a histidine at position 67 of the amino acid sequence, but not produced or may be produced at low levels on enzymatic digestion of bovine beta-casein variants that have a proline at position 67 of the amino acid sequence.

The term “regurgitation” means the act of bringing swallowed food back into the mouth.

The term “regurgitation episode” means a single incidence or event of regurgitation.

The term “frequency” with reference to a regurgitation episode means the number of regurgitation episodes over a period of time.

The term “variant” used in connection with a protein means a member of a set of highly similar proteins that are encoded by a single gene or gene family and are the result of genetic differences.

Any reference to the beta-casein in infant milk formula comprising a percentage by weight of a beta-casein variant means the percentage by weight of that variant of the total beta-casein in the infant milk formula. For example, the expression “the infant milk formula contains beta-casein comprising 100% by weight beta-casein A2” means that 100% of the total beta-casein content of the infant milk formula is beta-casein A2.

Unless defined otherwise, all technical and scientific terms used herein have the same meaning as commonly understood to one of ordinary skill in the art to which the inventions belong. Although any assays, methods, devices and materials similar or equivalent to those described herein can be used in the practice or testing of the invention, various assays, methods, devices and materials are now described.

It is intended that reference to a range of numbers disclosed herein (for example, 1 to 10) also incorporates reference to all related numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

As used in this specification, the words “comprises”, “comprising”, and similar words, are not to be interpreted in an exclusive or exhaustive sense. In other words, they are intended to mean “including, but not limited to”.

Those skilled in the art will appreciate that the invention described in this specification is susceptible to variations and modifications other than those specifically described. It is to be understood that the invention includes all such variations and modifications. The invention also includes all of the steps or features referred to or indicated in this specification, individually or collectively, and any and all combinations or any two or more of said steps or features.

The invention is not to be limited in scope by the specific examples described in this specification, which are intended for the purpose of exemplification only. Functionally equivalent products, compositions and methods are clearly within the scope of the invention.

Any reference to prior art documents in this specification is not to be considered an admission that such prior art is widely known or forms part of the common general knowledge in the field.

The invention relates to infant milk formula for infants containing the protein beta-casein and its use for reducing the frequency of regurgitation episodes. The beta-casein comprises less than 30% by weight of beta-casein variants having histidine at position 67 of the beta-casein amino acid sequence, which includes the A1, B, C, F, G, H1 and H2 variants.

The applicant has found an association between infant milk formula made from milk containing beta-casein variants that readily produce BCM-7 on digestion and a higher frequency of regurgitation episodes in infants relative to infant milk formula made from milk containing beta-casein variants that do not readily produce BCM-7 on digestion. The only difference in structure between the two types of beta-casein variant is that one type has a histidine at position 67 whereas the other type has a proline at position 67. This difference enables beta-casein A1 (and related variants) to be readily digested in the gut to produce the opioid peptide BCM-7, whereas the presence of proline at position 67 sterically constrains beta-casein A2 (and related variants) making any production of BCM-7 from beta-casein A2 very difficult. The release of BCM-7 from beta-casein A1 (and related variants) in the gut of infants is considered to cause increased regurgitation episodes in infants. Accordingly, the reduction or elimination of infant milk formula containing beta-casein A1, or any other beta-casein variant having histidine at position 67, primarily through the choice of milk used in the manufacture of the infant milk formula, from the diet of infants is desirable.

The production of beta-caseins in the milk of a bovine cow is determined by genetics. For example, a bovine cow homozygous for the A1 allele (A1A1) can produce only the A1 beta-casein variant in its milk. A bovine cow homozygous for the A2 allele (A2A2) can produce only the A2 beta-casein variant in its milk. Further, a bovine cow heterozygous for the beta-casein A1 and A2 genes (A1A2) is able produce both the A1 and A2 beta-casein variants in its milk.

It is well-established that bovine cows can be genotyped for the beta-casein gene. Cows that are genotyped and determined to be homozygous for beta-casein having proline at position 67 include, but are not limited to, A2A2 and A2I cows. Cows determined to be homozygous for beta-casein having histidine at position 67 include, but are not limited to, A1A1 and A1B cows. Cows determined to be heterozygous beta-casein at position 67 include, but are not limited to, A1A2, A1I and A2B cows. Those cows that are determined to be homozygous for beta-casein having proline at position 67 can be separated from all others to produce a herd capable of producing only beta-caseins having proline at position 67 in their milk. The manufacture of infant milk formula from this milk therefore provides infant milk formula comprising no beta-casein A1 or any other variant having histidine at position 67. Thus, if the primary source of beta-casein in the infant milk formula contains the A2 variant and no other variant, the dietary intake of the A1 variant by an infant from that infant milk formula is all but eliminated and the adverse effects of BCM-7 on infant crying can therefore also be expected to be reduced or even eliminated.

Accordingly, the invention is based on the reduction or elimination of beta-casein A1, and other beta-casein variants having histidine at positon 67 of the amino acid sequence, in the infant milk formula, and the promotion of beta-casein A2, and other beta-casein variants having proline at positon 67 of the amino acid sequence. This is achieved by ensuring that the beta-casein in the milk used to manufacture the infant milk formula is predominantly or exclusively beta-casein variants having proline at positon 67 of the amino acid sequence, preferably beta-casein A2.

The A1 and A2 beta-casein variants are the predominant variants in most dairy populations. The invention is therefore described further with reference primarily to beta-casein A1 and beta-casein A2.

Ideally, the beta-casein in the milk used to manufacture the infant milk formula comprises 100% beta-casein A2. However, infant regurgitation frequency may be reduced by any infant milk formula where the beta-casein comprises predominantly beta-casein A2 (or other beta-casein variants having proline at positon 67 of the amino acid sequence), for example, any amount between 70% and 100% by weight, including but not limited to 75%, 80%, 90%, 95%, 98% and 99% by weight.

It will be appreciated that since beta-casein variants having histidine at position 67 of the amino acid sequence have an adverse effect on regurgitation frequency in infants there is no specific lower limit to the amount of such beta-casein variants in the infant milk formula where the effect may be observed. For example, infant milk formula containing beta-casein comprising 49% by weight beta-casein A1 and 51% by weight beta-casein A2 is beneficial relative to infant milk formula containing beta-casein comprising 50% by weight beta-casein A1 and 50% by weight beta-casein A2. The applicant considers that infant milk formula manufactured from milk containing beta-casein comprising less than 30% by weight of any of beta-casein variants A1, B, C, F, G, H1 and H2, although preferably no amount of beta-casein variants A1, B, C, F, G, H1 and H2 at all, adequately reflects the invention.

Thus, it will be appreciated that the beta-casein composition of milk used to manufacture the infant milk formula may comprise less than 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% of any one or more of beta-casein variants A1, B, C, F, G, H1 and H2 or no amount of these beta-casein variants at all.

Support for the invention is found in Example 1 which describes a single-site, double-blind, randomised, controlled, parallel pilot study. The study investigated the relative effects of infant milk formula prepared from milk containing only variants having proline at position 67 of the beta-casein amino acid sequence (referred to as the A2 beta-casein-based infant formula), infant milk formula prepared from milk containing variants having proline at position 67 of the beta-casein amino acid sequence and variants having histidine at position 67 of the beta-casein amino acid sequence (referred to as the A1/A2 beta-casein-based infant formula), and breast milk, on infant gastrointestinal health, nutrition and growth. The inventors hypothesised that infant milk formula prepared from milk containing only variants having proline at position 67 of the beta-casein amino acid sequence would have similar effects to breast milk. The infant milk formula prepared from milk containing both types of beta-casein variant was found to contain the variants having histidine at position 67 and the variants having proline at position 67 in a ratio of approximately 40:60.

The inventors found, as early as the first week of the study, that infants consuming breast milk and those consuming the A2 beta-casein-based infant formula were less irritable, with significantly less frequent regurgitation episodes compared to infants consuming the A1/A2 beta-casein-based infant formula. This difference was maintained through to the end of the 8 week study period.

Pair-wise comparisons of between-group differences in the frequency of regurgitation episodes showed that significant differences were observed early on between the group of infants consuming breast milk and the group of infants consuming the A1/A2 beta-casein-based infant formula, as well as between the group of infants consuming the A1/A2 beta-casein-based infant formula and the group of infants consuming the A2 beta-casein-based infant formula. However, there were no observable differences between the group of infants consuming breast milk and the group of infants consuming the A2 beta-casein-based infant formula, which is consistent with the hypothesis.

This study represents the first clear scientific evidence of a link between the consumption by infants of infant milk formula containing beta-casein variants having histidine at position 67 (especially beta-casein A1) and regurgitation frequency. Through the applicant's finding, it is clear that the consumption of beta-caseins that readily produce BCM-7 by infants should be avoided. In practical terms, the benefits of the invention can be achieved by sourcing milk having a beta-casein content that is predominantly beta-casein A2 and producing infant milk formula derived from that milk, and making that infant milk formula available for the purpose of reducing regurgitation frequency in infants.

Support for the invention is also found in Example 2. The objective of this study was to compare and measure markers of infant comfort during the survey period between mixed-fed A2 IMF and A1/A2 IMF. The term “A2 IMF” means that the beta-casein content of the IMF comprises 100% by weight beta-casein variants having proline at position 67 of the beta-casein amino acid sequence, as determined by UPLC and mass spectrometry. The beta-casein content was determined to contain 17% by weight beta-casein I, with the remaining 87% by weight being almost all beta-casein A2. The term “A1/A2 IMF” means that the beta-casein content of the IMF is 30% by weight beta-casein variants having histidine at position 67 of the beta-casein amino acid sequence, as determined by UPLC and mass spectrometry and 70% by weight beta-casein variants having proline at position 67 of the beta-casein amino acid sequence, as determined by UPLC and mass spectrometry.

An Infant Gastrointestinal Symptom Questionnaire (IGSQ) was used to assess GI-related signs and symptoms, including times and duration of regurgitation episodes (spitting up/vomiting), in infants as observed by parents/caregivers. A total of 280 infants from Shanghai participated in the study, including 140 A2 IMF mixed-fed infants and 140 A1/A2 IMF mixed-fed infants. Mixed-fed infants were fed a mixture of IMF and human milk. They each received a daily consumption of IMF of more than 400 ml.

The individual item scores, five domain scores, and total IGSQ scores were similar between the groups at baseline. At week 2, the A2 IMF group had a significantly lower mean (SD) daily score than the control group for spitting up/vomiting (5.72 [1.45] vs 6.36 [1.40]), and a total IGSQ score (19.15 [3.74] vs 21.21 [3.30], all p<0.05). These scores were also significantly lower in the A2 IMF group than the A1/A2 IMF control group at week 4.

The data therefore clearly show that infants in the A2 group had reduced spitting up/vomiting episodes compared with those in the control group after 4 weeks. This was reflected in the parent/caregiver satisfaction surveys, which stated that infants in the A2 group had improved relief of gastrointestinal symptoms, including less vomiting after feeding, improved digestion and better stool characters.

The milk of cows can be tested for relative proportions of beta-casein variants containing proline or histidine at positon 67 of the amino acid sequence to determine whether the milk is suitable for use in preparing infant milk formula for the present invention. Alternatively, cows can be genetically tested for their ability to produce milk containing beta-casein A1 (or other beta-casein variants having histidine at positon 67 of the amino acid sequence) or tested for their ability to produce milk containing beta-casein A2 (or other beta-casein variants having proline at positon 67 of the amino acid sequence) or a combination of both. These techniques are well-known.

The present invention provides a solution that is comparatively easy to manage, i.e. ensuring that bovine milk used for preparing infant milk formula contains beta-casein that is predominantly beta-casein A2, or any of the A3, D, E and I variants, preferably 100% beta-casein A2.

The infant milk formula of the invention may be prepared using any known manufacturing process. Powdered infant milk formula may be manufactured by any standard method, typically using a dry blending process or a wet mixing/spray drying process. In a dry blending process, the ingredients are in a dehydrated powdered form and are mixed together to achieve a uniform blend of the macro and micronutrients necessary for a complete infant formula product.

In one example of the process, the blended product is passed through a sifter to remove oversize particles and extraneous material. The sifted product is then transferred to bags, totes or lined fibreboard drums for storage. In some cases, the powder may be transferred directly to a powder packaging line. At the packaging line, the powder is transferred to a filler hopper that feeds powder into a can filling line.

In one example of a wet blending/spray drying process, the ingredients are blended together, homogenised, pasteurised and spray dried to produce the powdered product. After pasteurisation, the liquid is usually homogenised and any heat sensitive micronutrients (e.g., vitamins, amino acids and fatty acids) are then added. The liquid may be concentrated by passing it through an evaporator or it may be pumped directly to a spray dryer. After spray drying, the product may be agglomerated to increase the particle size and to improve its solubility. In an alternative process, the milk may be dried by drum drying where milk is applied as a thin film to the surface of a heated drum. Freeze drying may also be used. The drying method and the heat treatment of the milk as it is processed alters the properties of the milk powder, such as its solubility in cold water, its flavour and its bulk density. The finished powder is passed through a sifter then transferred to bags, totes or silos for storage, or transferred directly to a powder packaging line.

Infant milk formula is typically in the form of a powder, but may also be in the form of a liquid concentrate or in ready-to-feed form. Water is mixed with powdered or liquid concentrate forms to prepare the infant milk formula ready for drinking by an infant.

The invention is further described with reference to the following examples. It will be appreciated that the invention as claimed is not intended to be limited in any way by this example.

### EXAMPLES

### Example 1: Infant Milk Formula Trial and Regurgitation Measurements I

### Study Design

The study was a single-site, double-blinded, randomised, controlled, parallel pilot study comprising 32 infants aged 40-60 days who were breast fed prior to the study, had normal electrocardiogram (ECG) and blood pressure during quiet respiration, and did not to take any medication, supplements, nutrition or other dairy products including acidophilus milk. The infant milk formula used in the study was either (1) prepared from bovine cows' milk containing beta-casein having histidine at position 67 and beta-casein having proline at position 67 in a ratio of approximately 40:60 (Group A1/A2), or (2) infant milk formula prepared from cows' milk containing 100% beta-casein having proline at position 67 (Group A2), in each case as confirmed by ultra-performance liquid chromatography and mass spectrometry.

Statistical analyses of study data used the statistical software package SAS version 9.3 (SAS Institute Inc., USA). All statistical tests of hypothesis were two-sided and performed at the 0.05 significance level. Mean and standard deviation (SD) of continuous variables and frequency (%) of categorical valuables at baseline and post-intervention were provided. Baseline subject characteristics including age, gender, height, body weight, head circumference and chest circumference for all enrolled subjects were summarised using mean and standard deviation (SD) for continuous outcomes and frequency and percentage for categorical measures. The differences among study groups were evaluated using analysis of variance (ANOVA) for continuous variables. Further pair-wise group comparison was then performed for outcomes with significant group difference according to the results from ANOVA. Bonfferoni correction was applied for multiple comparison. Paired t-test was used to evaluate the pre-post difference within each study group.

### Results

Summary statistics for baseline subject characteristics by study groups are shown in Table 1. Data are mean (standard deviation) for continuous variables or frequency (%) for categorical variables. Results from one-way ANOVA showed no significant difference among the three study groups, implicating that the randomisation was valid.

TABLE 1
Evaluation of Baseline Characteristics - Mean (SD) or Frequency (%),

| | | | | | | | | | | ANOVA |
|---|---|---|---|---|---|---|---|---|---|---|
| | | BF (n = 11) | | A1/A2 (n = 11) | | A2 (n = 11) | | Overall (n = 33) | | (p-value) |
| Gender | Male | 7 | (63.6%) | 6 | (54.5%) | 8 | (72.7%) | 21 | (63.6%) | / |
| | Female | 4 | (36.4%) | 5 | (45.5%) | 3 | (27.3%) | 12 | (36.4%) | |

| | | | | | | | | | |
|---|---|---|---|---|---|---|---|---|---|
| Age (day) | 48.2 | (5.2) | 46.4 | (5.4) | 47.4 | (8.2) | 47.3 | (6.2) | 0.802 |
| Weight (g) | 5397.3 | (589.7) | 5228.1 | (786.8) | 5308.2 | (652.1) | 5311.2 | (663.3) | 0.845 |
| Height (cm) | 57.8 | (2.0) | 56.3 | (2.8) | 57.6 | (2.1) | 57.2 | (2.4) | 0.304 |
| Head | 37.6 | (1.1) | 37.4 | (1.4) | 37.7 | (1.7) | 37.6 | (1.3) | 0.918 |
| Circumference | | | | | | | | | |
| (cm) | | | | | | | | | |
| Chest | 37.8 | (1.4) | 37.1 | (1.9) | 37.4 | (1.5) | 37.4 | (1.6) | 0.648 |
| Circumference | | | | | | | | | |
| (cm) | | | | | | | | | |

The summary statistics of the baseline and weekly average of daily milk regurgitation frequency for each study group are shown in Table 2. The two groups had similar baseline values for all daily life habit outcomes. Significant group difference was observed in regurgitation frequency from the first week after product intervention between the BF (breast fed) and A1 groups but not the BF and A2 groups.

TABLE 2
Summary of Weekly Average of Daily Life Habit Outcomes by Study
Group Mean (SD), p-value (ANOVA)

| | | | | A1/A2 | | | | Overall | | p-value |
|---|---|---|---|---|---|---|---|---|---|---|
| | Measurement | BF (n = 11) | | (n = 11) | | A2 (n = 10) | | (n = 32) | | (ANOVA) |
| Milk | Baseline | 2.36 | (1.29) | 2.45 | (1.04) | 2.20 | (1.03) | 2.34 | (1.10) | 0.873 |
| regurgitation | Week 1 | 2.12 | (0.85) | 2.88 | (0.37) | 2.53 | (0.61) | 2.51 | (0.70) | 0.033* |
| frequency | Week 2 | 1.97 | (0.80) | 2.79 | (0.43) | 2.13 | (0.77) | 2.30 | (0.75) | 0.022* |
| (times/day) | Week 3 | 1.85 | (0.69) | 2.73 | (0.49) | 2.03 | (0.48) | 2.21 | (0.67) | 0.003** |
| | Week 4 | 1.64 | (0.71) | 2.64 | (0.43) | 1.77 | (0.52) | 2.02 | (0.71) | <0.001*** |
| | Week 5 | 1.58 | (0.65) | 2.36 | (0.28) | 1.70 | (0.51) | 1.89 | (0.60) | 0.002** |
| | Week 6 | 1.45 | (0.67) | 2.39 | (0.47) | 1.50 | (0.55) | 1.79 | (0.71) | 0.001** |
| | Week 7 | 1.24 | (0.50) | 2.21 | (0.31) | 1.47 | (0.65) | 1.65 | (0.64) | <0.001*** |
| | Week 8 | 1.33 | (0.39) | 2.15 | (0.46) | 1.40 | (0.41) | 1.64 | (0.56) | <0.001*** |

*: p <0.05;
**: p <0.01;
***: p <0.001;
no symbol: p ≥0.05/3

Pair-wise comparison (Table 3) showed a significant difference between the Group A1/A2 and the breast fed (BF) group starting from week 1, and between Group A1/A2 and Group A2 starting from week 2, while there was no significant difference between Group A2 and the breast fed group throughout the study period.

TABLE 3
Pair-wise Comparison of Weekly Average of Daily Life Habit
Outcomes

| | | | Mean | 95% Cl | | |
|---|---|---|---|---|---|---|
| | Measurement | Groups | Difference | Lower | Upper | p-value |
| Milk | Baseline | BF vs. A1/A2 | −0.091 | −1.070 | 0.890 | 0.851 |
| regurgitation | | BF vs. A2 | 0.164 | −0.840 | 1.170 | 0.742 |
| | | A1/A2 vs. A2 | 0.255 | −0.750 | 1.260 | 0.609 |
| | Week 1 | BF vs. A1/A2 | −0.758 | −1.317 | −0.198 | 0.010* |
| | | BF vs. A2 | −0.412 | −0.986 | 0.161 | 0.152 |
| | | A1/A2 vs. A2 | 0.346 | −0.228 | 0.919 | 0.228 |
| | Week 2 | BF vs. A1/A2 | −0.818 | −1.414 | −0.222 | 0.009* |
| | | BF vs. A2 | −0.164 | −0.774 | 0.447 | 0.588 |
| | | A1/A2 vs. A2 | 0.655 | 0.044 | 1.265 | 0.037 |
| | Week 3 | BF vs. A1/A2 | −0.879 | −1.371 | −0.386 | 0.001** |
| | | BF vs. A2 | −0.185 | −0.690 | 0.320 | 0.460 |
| | | A1/A2 vs. A2 | 0.694 | 0.189 | 1.199 | 0.009** |
| | Week 4 | BF vs. A1/A2 | −1.000 | −1.495 | −0.505 | <0.001 |
| | | BF vs. A2 | −0.130 | −0.637 | 0.376 | 0.603 |
| | | A1/A2 vs. A2 | 0.870 | 0.363 | 1.376 | 0.001* |
| | Week 5 | BF vs. A1/A2 | −0.788 | −1.226 | −0.349 | 0.001 |
| | | BF vs. A2 | −0.124 | −0.574 | 0.325 | 0.576 |
| | | A1/A2 vs. A2 | 0.664 | 0.214 | 1.113 | 0.005** |
| | Week 6 | BF vs. A1/A2 | −0.939 | −1.436 | −0.443 | 0.001 |
| | | BF vs. A2 | −0.046 | −0.554 | 0.464 | 0.856 |
| | | A1/A2 vs. A2 | 0.894 | 0.385 | 1.403 | 0.001 |
| | Week 7 | BF vs. A1/A2 | −0.970 | −1.405 | −0.534 | <0.001 |
| | | BF vs. A2 | −0.224 | −0.671 | 0.222 | 0.313 |
| | | A1/A2 vs. A2 | 0.746 | 0.299 | 1.192 | 0.002** |
| | Week 8 | BF vs. A1/A2 | −0.818 | −1.186 | −0.451 | <0.001** |
| | | BF vs. A2 | −0.067 | −0.443 | 0.310 | 0.720 |
| | | A1/A2 vs. A2 | 0.752 | 0.375 | 1.128 | <0.001** |

*: p <0.005/3;
**: p <0.01;
***: p <0.001;
no symbol: p ≥0.05/3 with Bonferroni correction for multiple comparison

### Example 2: Infant Milk Formula Trial and Regurgitation Measurements II

### Study Design

The study was a randomised, open-label, prospective study and intended to evaluate the nutritive effects of A2 infant formula on tolerance in healthy term infants. A total of 280 healthy, term, mixed-fed infants (daily formula consumption more than 400 ml) were enrolled from Shanghai, including 140 A2 IMF mixed-fed infants and a control group of 140 A1/A2 IMF mixed-fed infants. Mixed-fed infants were fed a mixture of IMF and human milk, and received a daily consumption of IMF of more than 400 ml. The term “A2 IMF” means that the beta-casein content of the IMF comprises 100% by weight beta-casein variants having proline at position 67 of the beta-casein amino acid sequence, as determined by UPLC and mass spectrometry. The beta-casein content was determined to contain 17% by weight beta-casein I, with the remaining 87% by weight being almost all beta-casein A2. The term “A1/A2 IMF” means that the beta-casein content of the IMF is approximately 30% by weight beta-casein variants having histidine at position 67 of the beta-casein amino acid sequence, as determined by UPLC and mass spectrometry, and approximately 70% beta-casein variants having proline at position 67 of the beta-casein amino acid sequence, again as determined by UPLC and mass spectrometry.

The study duration was 56 days, before the introduction of complementary food. There were 4 visits (day 0, day 14, day 28 and day 56). At each visit, a spitting up/vomiting (regurgitation) questionnaire and a gastrointestinal tolerance questionnaire were completed. At the first visit, parents/guardians were given a “subject diary”, including a daily record of spitting up/vomiting frequency. All parents/guardians were given a “Subject Diary” and were instructed to bring the diary to each study visit for review. The frequency and volume of formula intake was recorded. Parents/guardians also recorded the presence of other food/liquid intake (including infant cereal, non-study formula, water, baby food, or other). An Infant Gastrointestinal Symptom Questionnaire (IGSQ) was used to assess GI-related signs and symptoms observed by parents/guardians in various domains including spitting up/vomiting as well as stooling, crying, flatulence and fussiness. The daily average of the domain scores during the past week of each visit were tabulated for each study group. The composite IGSQ scores (sum of all domain scores) were calculated for each study group and compared between study groups using ANCOVA with adjustment for baseline composite IGSQ score, age, gender, and the average amount of formula intake per day during the past week of each visit.

### Statistical Analysis

Descriptive statistics of study variables were provided by study group. Mean and standard deviation were summarised for normal distributed continuous variables; median and inter-quartile range were provided for non-normal variables; frequency and percentage were provided for categorical variables. A 0.05 level of significance was used for two-sided statistical testing. Prior to testing, distributional assumptions for the outcomes were assessed and transformations or nonparametric versions of the tests were used if deemed necessary. A 95% confidence level was used for confidence intervals. All analyses were conducted using the SAS software, version 9.4 (SAS Institute Inc., Cary, NC, USA).

### Results

Of the 280 eligible participants enrolled at baseline and initiated study formula intake, 29 withdrew early during the study (14 from the A2 group and 15 from the A1/A2 control group) resulting in a 10.36% overall dropout rate. The numbers of subjects that completed each study visit are listed by group in Table 4.

TABLE 4
Number of Subjects that Completed Each Visit

| Visit | A2 | Control |
|---|---|---|
| Visit 1 (baseline) | 140 | 140 |
| Visit 2 (end of week 2) | 136 | 138 |
| Visit 3 (end of week 4) | 130 | 134 |
| Visit 4 (end of week 8) | 126 | 125 |

Demographic and other baseline characteristics were summarised and compared for differences between study groups using one-way analysis of variance (ANOVA) for continuous variables with normal distribution, Kruskal-Wallis test for non-normal continuous variables, and chi-square/Fisher's Exact test for categorical variables, as shown in Table 5.

TABLE 5
Demographics and Baseline Characteristics

| | | | |
|---|---|---|---|
| | | | Group |
| | A2 | Control | Difference |
| Variable | (n = 140) | (n = 140) | p-value |

Demographics:

| | | | |
|---|---|---|---|
| Infant age at | 100.83 ± 15.01 | 99.28 ± 13.15 | 0.359 |
| baseline, days | | | |
| Gender, % | | | 0.281 |
| Male | 61 (43.57) | 71 (50.71) | |
| Female | 79 (56.43) | 69 (49.29) | |
| Weight at birth, g | 3345.16 ± 349.51 | 3350.92 ± 362.97 | 0.892 |
| Length at birth, cm | 49.87 ± 1.54 | 49.90 ± 1.76 | 0.871 |
| Head circumference | 34.18 ± 1.05 | 34.22 ± 1.00 | 0.757 |
| at birth, cm | | | |
| Gestational age, day | 39.00 ± 0.97 | 39.05 ± 0.98 | 0.670 |

### Infant Gastrointestinal Symptom Questionnaire (IGSQ)

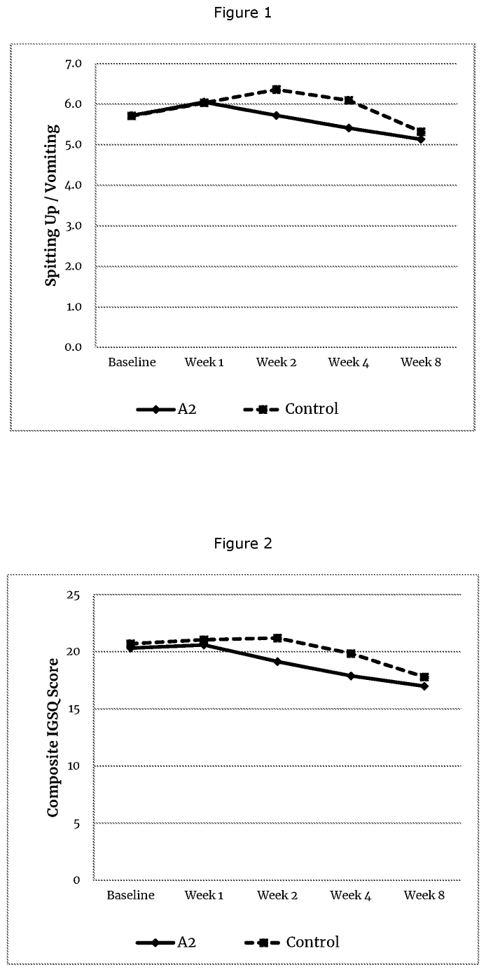

All individual item scores, spitting up/vomiting scores, and the total score of IGSQ were comparable between the two study groups at baseline. In week 2 and week 4, the A2 group showed significant lower average daily scores for spitting up/vomiting (p<0.0001 and p=0.0002). No significant group difference was observed for other scores in week 1 and week 8. See FIGS. 1 and 2.

TABLE 6
Average Daily Spitting Up/Vomiting and IGSQ Scores

| | | | | Group Difference | | |
|---|---|---|---|---|---|---|
| | | | | (A2 vs. Control) | | |
| | | | | Difference | | |
| | | | | of Least- | | |
| | Time | | | square Means | | |
| Variable | Point | A2 | Control | (95% CI) | | p-value |
| Spitting up/ | Baseline | 5.72 ± 1.51 | 5.71 ± 1.44 | 0.05 | (−0.30, 0.39) | 0.776 |
| vomiting | Week 1 | 6.05 ± 1.49 | 6.03 ± 1.33 | −0.01 | (−0.31, 0.30) | 0.959 |
| | Week 2 | 5.72 ± 1.45 | 6.36 ± 1.40 | −0.66 | (−0.98, −0.35) | <0.0001* |
| | Week 4 | 5.41 ± 1.26 | 6.09 ± 1.58 | −0.65 | (−0.99, −0.31) | 0.0002* |
| | Week 8 | 5.13 ± 1.19 | 5.32 ± 1.01 | −0.19 | (−0.46, 0.07) | 0.152 |

| Frequency | Baseline | 2 | (1, 2), | 2 | (1, 2), | / | 0.704 |
|---|---|---|---|---|---|---|---|
| of spit up ^{b} | | [1, 4] | | [1, 4] | | | |
| | Week 1 | 2 | (1.57, 2.43), | 2.14 | (1.86, 2.43), | / | 0.164 |
| | | [1, 5] | | [1, 3.86] | | | |
| | Week 2 | 1.93 | (1.29, 2.29), | 2.29 | (2, 2.57), | / | <0.0001* |
| | | [1, 5] | | [1, 3.29] | | | |
| | Week 4 | 1.71 | (1.14, 2.14), | 2 | (1.71, 2.43), | / | <0.0001* |
| | | [1, 3.57] | | [1, 4] | | | |
| | Week 8 | 1.64 | (1, 2), | 1.71 | (1.43, 2), | / | 0.037* |
| | | [1, 4] | | [1, 3.14] | | | |
| Volume of | Baseline | 1 | (1, 1), | 1 | (1, 1), | / | 0.680 |
| milk spit | | [1, 3] | | [1, 3] | | | |
| up ^{b} | Week 1 | 1.14 | (1, 1.43), | 1.14 | (1, 1.43), | / | 0.831 |
| | | [1, 5] | | [1, 2.57] | | | |
| | Week 2 | 1 | (1, 1.29), | 1.29 | (1, 1.43), | / | 0.004* |
| | | [1, 3] | | [1, 2.57] | | | |
| | Week 4 | 1 | (1, 1.29), | 1.14 | (1, 1.43), | / | 0.002* |
| | | [1, 2.29] | | [1, 3] | | | |
| | Week 8 | 1 | (1, 1.14), | 1 | (1, 1.29), | / | 0.133 |
| | | [1, 2.43] | | [1, 2.14] | | | |
| Discomfort | Baseline | 1 | (1, 2), | 1 | (1, 2), | / | 0.671 |
| when | | [1, 3] | | [1, 3] | | | |
| spitting up ^{b} | Week 1 | 1.43 | (1, 2), | 1.43 | (1.14, 1.86), | | 0.808 |
| | | [1, 3.14] | | [1, 3.29] | | | |
| | Week 2 | 1.29 ( | 1, 1.86), | 1.71 | (1.29, 2.07), | / | <0.0001* |
| | | [1, 3] | | [1, 4.57] | | | |
| | Week 4 | 1.29 | (1, 1.71), | 1.57 | (1.14, 2), | / | <0.0001* |
| | | [1, 3] | | [1, 5] | | | |
| | Week 8 | 1 | (1, 1.43), | 1.29 | (1, 1.57), | / | 0.036* |

| | | [1, 3] | [1, 2.71] | | | |
|---|---|---|---|---|---|---|
| Composite | Baseline | 20.34 ± 4.44 | 20.71 ± 3.95 | −0.25 | (−1.23, 0.73) | 0.615 |
| IGSQ score | | | | | | |
| | Week 1 | 20.61 ± 4.03 | 21.06 ± 3.25 | −0.28 | (−1.05, 0.49) | 0.473 |
| | Week 2 | 19.15 ± 3.74 | 21.21 ± 3.30 | −1.93 | (−2.69, −1.17) | <0.0001* |
| | Week 4 | 17.90 ± 3.49 | 19.86 ± 3.73 | −1.72 | (−2.57, −0.88) | <0.0001* |
| | Week 8 | 17.00 ± 3.11 | 17.80 ± 2.78 | −0.68 | (−1.37, 0.02) | 0.058 |

CI: confidence interval.

Unless otherwise stated, data presented are mean±standard deviation. Group differences were evaluated using analysis of covariance, adjusted for age, gender and average daily amount of formula intake in the past week. Post-intervention analysis also adjusted for baseline scores. *: p<0.05
^{a.} Baseline data were based on 24-hour recall of symptoms of the previous day. Group differences were evaluated using analysis of covariance, adjusted for age and gender.
^{a.} Data presented are median (1^{st} quartile, 3^{rd} quartile) and range. Group differences were evaluated using Kruskal Wallis test.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

1. A method for reducing the frequency of regurgitation episodes in an infant by providing infant milk formula to the infant for ingestion, wherein the milk used in the manufacture of the infant milk formula contains beta-casein comprising less than 30% by weight of beta-caseins having histidine at position 67 of the beta-casein amino acid sequence.

2. A method as claimed in claim 1, wherein the beta-casein comprises less than 30% by weight of any one or more of beta-casein variants A1, B, C, F, G, H1 and H2.

3. A method as claimed in claim 1 or claim 2, wherein the beta-casein comprises less than 25% by weight of beta-casein A1.

4. A method as claimed in any one of claims 1 to 3, wherein the beta-casein comprises less than 10% by weight beta-casein A1.

5. A method as claimed in any one of claims 1 to 4, wherein the beta-casein comprises less than 5% by weight beta-casein A1.

6. A method as claimed in any one of claims 1 to 5, wherein the beta-casein comprises less than 1% by weight beta-casein A1.

7. A method as claimed in any one of claims 1 to 6, wherein the beta-casein comprises no beta-casein A1.

8. A method as claimed in any one of claims 1 to 7, wherein the beta-casein comprises at least 70% by weight of beta-caseins having proline at position 67 of the beta-casein amino acid sequence.

9. A method as claimed in any one of claims 1 to 8, wherein the beta-casein comprises at least 70% by weight of any one or more of beta-casein variants A2, A3, D, E and I.

10. A method as claimed in any one of claims 1 to 9, wherein the beta-casein comprises at least 75% by weight beta-casein A2.

11. A method as claimed in any one of claims 1 to 10, wherein the beta-casein comprises at least 90% by weight beta-casein A2.

12. A method as claimed in any one of claims 1 to 11, wherein the beta-casein comprises at least 95% by weight beta-casein A2.

13. A method as claimed in any one of claims 1 to 12, wherein the beta-casein comprises at least 99% by weight beta-casein A2.

14. A method as claimed in any one of claims 1 to 13, wherein the beta-casein comprises 100% beta-casein A2.

15. A method as claimed in any one of claims 1 to 14, wherein the infant has an age less than six months or less than three months.

16. A method for reducing the frequency of regurgitation episodes in an infant by feeding infant milk formula to the infant, wherein the milk used in the manufacture of the infant milk formula contains beta-casein comprising less than 30% by weight of beta-caseins having histidine at position 67 of the beta-casein amino acid sequence.

17. A method for reducing the frequency of regurgitation episodes in an infant having a diet comprising infant milk formula where the infant milk formula contains beta-casein comprising at least 30% by weight of beta-caseins having histidine at position 67 of the beta-casein amino acid sequence, by replacing the infant milk formula with a replacement infant milk formula manufactured from milk containing beta-casein comprising less than 30% by weight of beta-caseins having histidine at position 67 of the beta-casein amino acid sequence.

18. A method for reducing the frequency of regurgitation episodes in an infant having a diet comprising a first infant milk formula, wherein the first infant milk formula is replaced with a second infant milk formula in the diet of the infant;
wherein the first infant milk formula contains beta-casein comprising at least 30% by weight of beta-caseins having histidine at position 67 of the beta-casein amino acid sequence; and
wherein the second infant milk formula is manufactured from milk containing beta-casein comprising less than 30% by weight of beta-caseins having histidine at position 67 of the beta-casein amino acid sequence.

19. Infant milk formula for reducing the frequency of regurgitation episodes in an infant which infant milk formula is manufactured from milk containing beta-casein comprising less than 30% by weight of beta-caseins having histidine at position 67 of the beta-casein amino acid sequence.

20. The use of bovine milk in the manufacture of infant milk formula for reducing the frequency of regurgitation episodes in an infant, wherein the milk contains beta-casein comprising less than 30% by weight of beta-caseins having histidine at position 67 of the beta-casein amino acid sequence.
